SEQUENCE LISTING (12 sequences)

SEQ ID NO: 1 - protein, 67 aa, Staphylococcus aureus
MVDNKFNKEQQNAFYEILHLPNLNKEQRNAFIQSLKDDPSQSANLLAEAKKLNDAQAPKGSHHHHHH

SEQ ID NO: 2 - protein, 33 aa, Staphylococcus aureus
FNKEQQNAFYEILHLPNLNKEQRNAFIQSLKDD

SEQ ID NO: 3 - DNA, 204 bp, Staphylococcus aureus
atggtggacaataaattcaacaaggagcaacagaacgccttttatgaaatccttcatttgccaaaccttaacgaggaacagaggaatgcgtttattcaatcacttaaggatgatccaagtcaatcggccaacctgttggcggaggccaaaaaattaaatgacgcacaagcgcctaaaggatcccaccaccatcatcatcattga

SEQ ID NO: 4 - DNA, 60 bp, Artificial Sequence
  L18TAG
ggagcaacagaacgccttttatgaaatctagcatttgccaaaccttaacgaggaacagag

SEQ ID NO: 5 - DNA, 60 bp, Artificial Sequence
  H19TAG
gcaacagaacgccttttatgaaatcctttagttgccaaaccttaacgaggaacagaggaa

SEQ ID NO: 6 - DNA, 60 bp, Artificial Sequence
  E25TAG
tgaaatccttcatttgccaaaccttaactaggaacagaggaatgcgtttattcaatcact

SEQ ID NO: 7 - DNA, 60 bp, Artificial Sequence
  E26TAG
aatccttcatttgccaaaccttaacgagtagcagaggaatgcgtttattcaatcacttaa

SEQ ID NO: 8 - DNA, 60 bp, Artificial Sequence
  R28TAG
tcatttgccaaaccttaacgaggaacagtagaatgcgtttattcaatcacttaaggatga

SEQ ID NO: 9 - DNA, 60 bp, Artificial Sequence
  N29TAG
tttgccaaaccttaacgaggaacagaggtaggcgtttattcaatcacttaaggatgatcc

SEQ ID NO: 10 - DNA, 60 bp, Artificial Sequence
  FPheKRS-f
tcacaaaggaggtgcggccgcatggataaaaaaccattagatgttttaatatctgcgacc

SEQ ID NO: 11 - DNA, 60 bp, Artificial Sequence
  FPheKRS-r
gaccgtttaaacgcggccgcttatagattggttgaaatcccattatagtaagattcggac

SEQ ID NO: 12 - protein, 14 aa, Artificial Sequence
  Cyclic Fclll-FPheK peptide
DDCAWKLGELVWCT

(19) United States
(12) Patent Application Publication
(10) Pub. No.: US 2021/0130395 A1
(43) Pub. Date: May 6, 2021

(54) PROXIMITY INDUCED SITE-SPECIFIC ANTIBODY CONJUGATION

(71) Applicant: William Marsh Rice University, Houston, TX (US)

(72) Inventors: Han XIAO, Houston, TX (US); Chenfei YU, Houston, TX (US); Juan TANG, Houston, TX (US)

(73) Assignee: William Marsh Rice University, Houston, TX (US)

(21) Appl. No.: 17/054,312

(22) PCT Filed: May 10, 2019
(86) PCT No.: PCT/US2019/031836

§ 371 Date: Nov. 10, 2020

Related U.S. Application Data
(60) Provisional application No. 62/670,675, filed on May 11, 2018.

(51) Int. Cl. C07K 1/13; C07K 16/00; C07K 16/32; G01N 33/533

(52) U.S. Cl. CPC C07K 1/13; C07K 16/00; C07K 16/32; C07K 2317/24; C07K 2317/524; C07K 2317/526; C07K 2317/21; G01N 33/533

(57) ABSTRACT

The present disclosure provides methods for proximity-induced antibody conjugation of target agents).

This application claims the benefit of U.S. Provisional Patent Application No. 62/670,675, filed May 11, 2018, which is incorporated herein by reference in its entirety.

### INCORPORATION OF SEQUENCE LISTING

The sequence listing that is contained in the file named “RICEP044WO.txt”, which is 3.35 KB (as measured in Microsoft Windows) and was created on May 10, 2019, is filed herewith by electronic submission and is incorporated by reference herein.

### BACKGROUND

### 1. Field

The disclosure relates generally to the field of molecular biology. More particularly, it concerns methods of conjugating agent(s) to an antibody.

### 2. Related Art

Monoclonal antibodies with excellent selectivity and a broad collection of targets are extensively used as affinity reagents in many biological applications, from in vitro assays to disease diagnostics to targeted therapies. These applications often require the modification of antibodies by various chemical molecules (e.g., fluorophores, drugs, nanoparticles) or biological reagents (e.g., enzymes, cytokines, antibodies). To covalently label antibodies, various methods have been developed, most commonly involving nonspecific acylation of lysine residues with highly reactive esters and alkylation of cysteine residues with maleimides. The resulting products are heterogeneous antibody conjugates that cannot be further purified. Antibodies derived from such heterogeneous modification may suffer from diminished binding affinity and therapeutic index due to a lack of control over the modification ratio and site [1-3].

With advances in the fields of bioorthogonal chemistry and protein engineering, several strategies have been developed for preparing site-specific antibody conjugates [4]. These include THIOMAB™, which affords ultra-reactive cysteine residues for conjugation [5]; SMARTag™, which genetically encodes a peptide tag for further enzymatic modification [6-8]; and the SiteClick™ labeling system, which introduces an unnatural sugar and noncanonical amino acid (ncAA) technology that enables site-specific incorporation of the 21st amino acid with a distinct reactive moiety [9-12]. In general, current site-specific antibody-labeling methods first require the site-specific introduction of a unique reactive moiety into antibodies, followed by selective modification using bioorthogonal chemistry. However, the site-specific installation of a bioorthogonal functionality requires a certain amount of antibody engineering, which is time-consuming, expensive, and may result in low yield. Thus, there is an unmet need for a new platform for rapid, efficient, site-specific labeling of antibodies.

### SUMMARY

In a first embodiment, the present disclosure provides methods for proximity-induced site-specific conjugation of a target agent to an antibody comprising providing an affinity compound having a proximity-reactive motif, wherein the affinity compound is conjugated to the target agent; and bringing the affinity compound into proximity of the antibody for a sufficient period of time to covalently link the affinity compound to said antibody. In some aspects, the proximity-reactive motif comprises a non-canonical amino acid (ncAA).

In some aspects, the affinity compound is a small molecule, DNA, RNA, peptide, protein or a derivative thereof. In some aspects, the RNA is an RNA aptamer. In certain aspects, obtaining the affinity compound is produced by solid-phase synthesis or recombinant expression. In some aspects, the affinity compound is further defined as an antibody-binding compound comprising a proximity reaction motif.

In certain aspects, the ncAA has the ability to crosslink with an amino acid residue of said antibody. In particular aspects, the amino acid residue is histidine, serine, threonine, tryptophan, tyrosine, lysine or cysteine. In some aspects, the ncAA has a reactive halide, aryl ketone, Michael acceptor, aryl isothiocyanate, or aryl carbamate side chain. In certain aspects, the ncAA contains a 4-fluorophenyl, acryloyl, fluorosulfate, sulfonyl fluoride, or reactive halide side chain(s). In certain aspects, the ncAA is 4-fluorophenyl carbamate lysine (FPheK), phenyl carbamate lysine (PheK), N-acryloyl-lysine (AcrK), 2-amino-6-(6-bromohexanamido)hexanoic acid (BrC6K), fluorosulfate-L-tyrosine (FSY), 2-amino-3-(4-(3-bromopropoxy)phenyl)propanoic acid (BprY), sulfonyl fluoride phenylalanine, or N-fluoroacetyllysine (FAcK). In particular aspects, the ncAA is FPheK.

In some aspects, the affinity compound exhibits binding for the fragment crystallizable (Fc) region, an antigen-binding (Fab) region, or hinge region of said antibody. In particular aspects, the affinity compound exhibits binding for the CH2 or CH3 region of said antibody. In specific aspects, the affinity compound exhibits binding for the CH2-CH3 junction of said antibody.

In some aspects, the affinity compound is a peptide derived from protein A (e.g., Z domain), protein G, or antibody-binding peptides evolved via phage display (e.g., FcIII). In particular aspects, the affinity compound is the B domain of protein A (FB protein) from Staphylococcus aureus, such as SEQ ID NO:1 (MVDNKFNKEQQNAFYEILHLPNLNXEQRNAFIQSLKDDPSQSANLLAEAKKLNDAQ APKGSHHHHHH (X=MMT-Lys)). In some aspects, the ncAA is inserted at residue 25 of the FB protein. In certain aspects, FPheK is inserted at residue 25 of the FB protein (FB-E25FPheK). In some aspects, the affinity compound is a peptide of SEQ ID NO:1 (e.g., a peptide having 90%, 91%, 92%, 93%, 94%, 95%, 96%, 97%, 98%, or 99% sequence identity to SEQ ID NO:1) or a fragment thereof, such as a peptide of 66, 65, 60, 55, 50, 45, 40, 35, 34, 33, 32, 31, 30, 29, 28, 27, 26, 25, or less amino acids in length. In particular aspects, the affinity compound is a peptide of SEQ ID NO:2 (FNKEQQNAFYEILHLPNLNXEQRNAFIQSLKDD (X=MMT-Lys)). In some aspects, the affinity compound is synthesized vis Fmoc-based solid-phase peptide synthesis, such as ssFB.

In certain aspects, the antibody is an IgG, IgM, IgA, IgE, or antigen binding fragment thereof. In some aspects, the antibody is a Fab′, a F(ab′)2, a F(ab′)3, a monovalent scFv, a bivalent scFv, a single domain antibody, or nanobody. In some aspects, the antibody is a human antibody or an antibody from another species. In specific aspects, the antibody is trastuzumab.

In certain aspects, the covalent linking has an efficiency of at least 40%, such as at least 50%, at least 60%, at least 70%, at least 80%, at least 90%, at least 95%, or at least 99%. In some aspects, the covalent linking does not comprise enzymatic treatment. In particular aspects, the covalent linking of the affinity peptide occurs without the use of other agents or the application of additional treatments.

In some aspects, the target agent is an imaging agent and/or therapeutic agent. In certain aspects, the therapeutic agent is a toxin or chemotherapeutic agent. In some aspects, the imaging agent is a fluorophore or radionuclide. In some aspects, the imaging agent is a PET probe or MRI probe. In certain aspects, the target agent is a drug, small molecule, DNA, RNA, small molecule, protein, peptide, enzyme, nanoparticle, virus, cell, saccharide, antibody or fragment thereof. In some aspects, the antibody is an Fc, Fab, scFv, single-domain antibody, or κ-light chain. In additional aspects, more than one target agent is conjugated to said antibody. In some aspects, 2, 3, 4, or 5 target agents are conjugated to the antibody. In some aspects, the target agents are conjugated to the antibody at different sites.

Further provided herein is a composition comprising an ncAA linker conjugated to a target agent. In additional aspects, the composition further comprises an antibody. In some aspects, the composition is produced according to the present embodiments and aspects thereof.

In some aspects, the affinity compound is a small molecule, DNA, RNA, peptide, or protein. In certain aspects, obtaining the affinity compound is produced by solid-phase synthesis or recombinant expression. In some aspects, the affinity compound is further defined as an antibody-binding compound comprising a proximity reaction motif. In some aspects, the affinity peptide has a length of 10-60 amino acids, such as 30-60 amino acids, such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or more amino acids.

In certain aspects, the ncAA has the ability to crosslink with an amino acid residue of said antibody. In particular aspects, the amino acid residue is lysine or cysteine. In some aspects, the ncAA has a reactive halide, aryl ketone, Michael acceptor, aryl isothiocyanate, or aryl carbamate side chain. In certain aspects, the ncAA contains a 4-fluorophenyl, acryloyl, fluorosulfate, sulfonyl fluoride, or reactive halide side chain(s). In certain aspects, the ncAA is 4-fluorophenyl carbamate lysine (FPheK), phenyl carbamate lysine (PheK), N-acryloyl-lysine (AcrK), 2-amino-6-(6-bromohexanamido)hexanoic acid (BrC6K), fluorosulfate-L-tyrosine (FSY), 2-amino-3-(4-(3-bromopropoxy)phenyl)propanoic acid (BprY), sulfonyl fluoride phenylalanine, or N-fluoroacetyllysine (FAcK).

In some aspects, the affinity compound exhibits binding for the fragment crystallizable (Fc) region, an antigen-binding (Fab) region, or hinge region of said antibody. In particular aspects, the affinity compound exhibits binding for the CH2 or CH3 region of said antibody. In specific aspects, the affinity compound exhibits binding for the CH2-CH3 junction of said antibody.

In some aspects, the affinity compound is a peptide derived from protein A (e.g., Z domain), protein G, or antibody-binding peptides evolved via phage display (e.g., FcIII). In particular aspects, the affinity compound is the B domain of protein A (FB protein) from Staphylococcus aureus. In some aspects, the ncAA is inserted at residue 25 of the FB protein. In certain aspects, FPheK is inserted at residue 25 of the FB protein (FB-E25FPheK).

In certain aspects, the antibody is an IgG, IgM, IgA, or antigen binding fragment thereof. In some aspects, the antibody is a Fab′, a F(ab′)2, a F(ab′)3, a monovalent scFv, a bivalent scFv, or a single domain antibody. In some aspects, the antibody is a human antibody or an antibody from another species. In specific aspects, the antibody is trastuzumab.

In some aspects, the target agent is an imaging agent and/or therapeutic agent. In certain aspects, the therapeutic agent is a toxin or chemotherapeutic agent. In some aspects, the imaging agent is a fluorophore or radionuclide. In some aspects, the imaging agent is a PET probe or MRI probe. In certain aspects, the target agent is a drug, small molecule, DNA, RNA, small molecule, protein, peptide, enzyme, nanoparticle, virus, cell, saccharide, antibody or fragment thereof. In some aspects, the antibody is an Fc, Fab, scFv, single-domain antibody, or κ-light chain. In additional aspects, more than one target agent is conjugated to said antibody. In some aspects, 2, 3, 4, or 5 target agents are conjugated to the antibody. In some aspects, the target agents are conjugated to the antibody at different sites.

A further embodiment provides a pharmaceutical composition comprising the composition of the embodiments and aspects thereof and a pharmaceutically acceptable buffer, diluent or excipient.

In another embodiment there is provided a method of imaging and/or treating a disease in a subject comprising administering an effective amount of a conjugated antibody of the embodiments, a pharmaceutical composition of the embodiments, or a conjugated antibody produced according to the embodiments and aspects thereof, to the subject.

In a further embodiment there is provided a composition comprising a conjugated antibody of the embodiments for an in vitro assay, such as an assay for protein detection. In some aspects, the assay is a western blot, flow cytometry, immunofluorescence, immunoprecipitation, ELISA, or other assay. In some aspects, the antibody is an antibody-HRP conjugate or antibody fluorophore conjugate.

In another embodiment, there is provided a method for producing a FPheK-labeled FB affinity peptide comprising synthesizing a truncated FB peptide with a monomethoxytrityl (MMT) protection group using Fmoc-based solid-phase peptide synthesis; selectively removing the MMT protection group using acetic acid; and reacting the truncated FB peptide with 4-fluorophenyl chloroformate, thereby producing the FPheK-labeled FB affinity peptide. In some aspects, the MMT protection is at residue 25 of the truncated FB peptide. In certain aspects, solid-phase synthesis comprises stepwise synthesis starting from rink amide resin. In some aspects, the truncated FB peptide is N-terminal acetylated. In certain aspects, the acetic acid is 10% acetic acid. In some aspects, the truncated FB peptide comprise or consists of SEQ ID NO:2. In certain aspects, the method further comprises lyophilizing the FPheK-labeled FB affinity peptide. In some aspects, the method further comprises denaturing the FPheK-labeled FB affinity peptide.

denaturing comprises using urea.

It is contemplated that any method or composition described herein can be implemented with respect to any other method or composition described herein. For example, a compound synthesized by one method may be used in the preparation of a final compound according to a different method.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D: Preparation and characterization of the Tras-FB conjugate. (FIG. 1A) FPheK allowing for crosslinking to a proximal lysine residue will be introduced into FB protein derived from protein A. The resulting adaptive peptides can be used to label antibodies upon binding. (FIG. 1B) Structures of lysine analogs that can crosslink with the lysine residue. (FIG. 1C) The structure of the B domain from Staphylococcus protein A (FB protein) interaction with the Fc fragment is also shown (PDB: 1FC2). (FIG. 1D) SDS-PAGE analysis of crosslinked mixtures in the presence (left) and absence (right) of the reducing reagents.

FIGS. 2A-2D: Proximity-induced ligation between FB proteins and Tras. (FIG. 2A) Reducing SDS-PAGE analysis of a reaction of FB-E25FPheK with Tras for 0, 6, 12, 24, and 48 h (left) and at 2:1, 1:1, 1:2, 1:4, and 1:8 ratios. (FIG. 2B) Reducing SDS-PAGE analysis of the reaction of Tras and FB-E25FPheK, FB-E25AcrK and FB-E25BrC6K mutants. (FIG. 2C) SDS-PAGE analysis of the FB-E25FPheK mutant, Tras, and Tras/FB-E25FPheK (conj.) under non-reducing conditions, visualized by Coomassie staining (FIG. 2D) Mass spectrometry analysis of Tras and Tras/FB-E25FPheK.

FIGS. 3A-3D: (FIG. 3A) Reducing SDS-PAGE analysis of various IgG subclasses alone or after conjugation with the FB-E25FPheK mutant. (FIG. 3B) Preparation of the Tras-488 conjugate. (FIG. 3C) SDS-PAGE analysis of Tras and Tras-488, visualized by Coomassie staining (left) and UV transillumination (right). (FIG. 3D) Binding of Tras in SK-BR-3 and MDA-MB-468 cells visualized by confocal microscopy. Cells were incubated with 30 nM Tras-488 in media for 30 min at 37° C. and stained with DilC18 and Hoechst nuclear stain. Scale bar=50 μm.

FIG. 4: ESI-MS spectra of FB proteins.

FIG. 5: Affinity peptides for proximity-induced site-specific antibody conjugation.

FIGS. 6A-6B: (FIG. 6A) Schematic of antibody conjugate preparation. (FIG. 6B) Mass spectrometry analysis of the antibody conjugate prepared.

FIG. 7: Schematic of antibody conjugate preparation using FcIII as an affinity peptide.

FIGS. 8A-8C: On-resin reaction for Lys-modified antibody affinity peptide. (FIG. 8A) MMT (Monomethoxytrityl) protection group was first removed by mild acetic solution (AcOH:TFE:DCM=1:2:7) (FIG. 8B) on-resin reaction between the exposed free amine and 4-fluorophenyl chloroformate (FIG. 8C) TFA cleaves peptide from resin and removes the protection groups.

FIGS. 9A-9B: Characterization of solid-phase synthesized truncated FB peptide (ssFB) and Tras-ssFB conjugate. (FIG. 9A) Mass spectrometry analysis of ssFB-FPheK. (FIG. 9B) reducing SDS-PAGE analysis of Tras and Tras-ssFB conjugate.

### DESCRIPTION OF ILLUSTRATIVE EMBODIMENTS

Site-specific antibody conjugates with a well-defined structure and superb therapeutic index are of great interest for basic research, disease diagnostics, and therapy. Accordingly, in certain embodiments, the present disclosure provides methods for proximity-induced antibody conjugation. The method can enable site-specific covalent bond formation between functional moieties and antibodies without antibody engineering. The utility of this approach was demonstrated in the present studies by site-specific conjugation of a green fluorophore to a antibody and in vitro characterization of its activity.

Specifically, the present methods may comprise proximity-induced reactivity between an ncAA and a nearby antibody residue, such as a lysine or cysteine. The proximity-induced antibody conjugation, referred to herein pClick, enables a covalent bond formation between functional moieties and a defined residue, such as lysine, of antibodies without performing antibody engineering. This conjugation method can allow a rapid and efficient site-specific functionalization of immunoglobulin G from different species and subclasses.

The present methods can be used to conjugate a variety of molecules, including drugs, fluorophores, DNA, RNA, enzymes, antibodies, nanoparticles, carbohydrates, peptide, inhibitors, and/or viruses to antibodies without doing antibody engineering.

### I. Proximity-Induced Antibody Conjugation

The present methods can comprise proximity-induce site-specific conjugation of a target agent to an antibody using an affinity compound. The affinity compound may be any compound with a proximity motif for the antibody. The compound may be a small molecule, DNA, RNA, peptide, or protein. The affinity compound, such as the peptide, can be prepared using solid-phase synthesis or recombinant expression. For example, the affinity compound may be a B domain (Fb) protein of Staphyloccocus aureus or a RNA aptamer, such as an anti-Fc aptamer.

### A. Therapeutic or Imaging Agents

The antibodies of the present disclosure may be conjugated to a target agent, such as a therapeutic agent, cell-targeting agent and/or imaging agent.

Examples of target agents that may be conjugated to the present antibodies include exogenous materials that do not exist naturally in virions (originate from an external source), such as, but not limited to, nucleic acid molecules such as DNA (both nuclear and mitochondrial), RNA such as mRNA, tRNA, miRNA, and siRNA, aptamers and other nucleic acid-containing molecules, peptides, proteins, ribozymes, carbohydrates, polymers, therapeutics, small molecules and the like. In particular aspects, the heterologous sequence may be a peptide, nucleic acid, antibody, or fragment thereof. The nucleic acid may be an inhibitory nucleic acid, such as siRNA, shRNA, or miRNA.

Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine ¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

The target agent may be an amino acid sequence less than 200 amino acids, such as less than 50 amino acids. The length of the peptide may be about 5-10 or 10-20 amino acids, such as 20-30, 30-40, or 40-50.

A “therapeutic agent” as used herein refers to any agent that can be administered to a subject for the purpose of obtaining a therapeutic benefit of a disease or health-related condition. For example, antibodies conjugated to a therapeutic agent may be administered to a subject for the purpose of reducing the size of a tumor, reducing or inhibiting local invasiveness of a tumor, or reducing the risk of development of metastases.

A “diagnostic agent” or “imaging agent” (referred to interchangeably) as used herein refers to any agent that can be administered to a subject for the purpose of diagnosing a disease or health-related condition in a subject. Diagnosis may involve determining whether a disease is present, whether a disease has progressed, or any change in disease state.

The therapeutic or diagnostic agent may be a small molecule, a peptide, a protein, a polypeptide, an antibody, an antibody fragment, a DNA, or an RNA.

The term “siRNA” (short interfering RNA) refers to short double stranded RNA complex, typically 19-28 base pairs in length. In other words, siRNA is a double-stranded nucleic acid molecule comprising two nucleotide strands, each strand having about 19 to about 28 nucleotides (i.e., about 19, 20, 21, 22, 23, 24, 25, 26, 27, or 28 nucleotides). The complex often includes a 3′-overhang. siRNA can be made using techniques known to one skilled in the art and a wide variety of siRNA is commercially available from suppliers such as Integrated DNA Technologies, Inc. (Coralville, Iowa).

A “microRNA (miRNA)” is short, non-coding RNAs that can target and substantially silence protein coding genes through 3′-UTR elements. miRNAs can be approximately 21-22 nucleotides in length and arise from longer precursors, which are transcribed from non-protein-encoding genes.

The therapeutic agent may be an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinase inhibitor, a proteolytic protein, a proteolytic protein inhibitor, a semaphorin, a semaphorin receptor, a serotonin transport protein, a serotonin uptake inhibitor, a serotonin receptor, a serpin, a serpin receptor, or a tumor suppressor. The therapeutic agent may be a chemotherapeutic (e.g., alkylating agents, antimetabolites, antitumor antibiotics, mitotic inhibitors, or nitrosoureas) or radiotherapeutic.

The therapeutic agent may be BDNF, CNTF, CSF, EGF, FGF, G-SCF, GM-CSF, gonadotropin, IFN, IFG-1, M-CSF, NGF, PDGF, PEDF, TGF, TGF-B2, TNF, VEGF, prolactin, somatotropin, XIAP1, IL-1, IL-2, IL-3, IL-4, IL-5, IL-6, IL-7, IL-8, IL-9, IL-10, IL- 10(187A), viral IL-10, IL-11, IL-12, IL-13, IL-14, IL-15, IL-16, IL-17, or IL-18.

The therapeutic agent, such as the peptide, antibody, or RNAi, may be specific to a target gene. In some aspects, the target agent is an antibody, such as to produce a bispecific antibody. A target gene generally means a polynucleotide comprising a region that encodes a polypeptide, or a polynucleotide region that regulates replication, transcription or translation or other processes important to expression of the polypeptide, or a polynucleotide comprising both a region that encodes a polypeptide and a region operably linked thereto that regulates expression. The targeted gene can be chromosomal (genomic) or extrachromosomal. It may be endogenous to the cell, or it may be a foreign gene (a transgene). The foreign gene can be integrated into the host genome, or it may be present on an extrachromosomal genetic construct such as a plasmid or a cosmid. The targeted gene can also be derived from a pathogen, such as a virus, bacterium, fungus or protozoan, which is capable of infecting an organism or cell. Target genes may be viral and pro-viral genes that do not elicit the interferon response, such as retroviral genes. The target gene may be a protein-coding gene or a non-protein coding gene, such as a gene which codes for ribosomal RNAs, splicosomal RNA, tRNAs, etc.

Any gene being expressed in a cell can be targeted. Preferably, a target gene is one involved in or associated with the progression of cellular activities important to disease or of particular interest as a research object. Thus, by way of example, the following are classes of possible target genes that may be used in the methods of the present disclosure to modulate or attenuate target gene expression: developmental genes (e.g., adhesion molecules, cyclin kinase inhibitors, Wnt family members, Pax family members, Winged helix family members, Hox family members, cytokines/lymphokines and their receptors, growth or differentiation factors and their receptors, neurotransmitters and their receptors), tumor suppressor genes (e.g., APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, ras, MMAC1, FCC, MCC, FUS1, Gene 26 (CACNA2D2), PL6, Beta* (BLU), Luca-1 (HYAL1), Luca-2 (HYAL2), 123F2 (RASSF1), 101F6, Gene 21 (NPRL2), or a gene encoding a SEM A3 polypeptide), pro-apoptotic genes (e.g., CD95, caspase-3, Bax, Bag-1, CRADD, TSSC3, bax, hid, Bak, MKP-7, PARP, bad, bc1-2, MST1, bbc3, Sax, BIK, and BID), cytokines (e.g., GM-CSF, G-CSF, IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32 IFN-α, IFN-β, IFN-γ, MIP-1α, MIP-1β, TGF-β, TNF-α, TNF-β, PDGF, and mda7), oncogenes (e.g., ABLI, BLC1, BCL6, CBFA1, CBL, CSFIR, ERBA, ERBB, EBRB2, ETS1, ETS1, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3 and YES), and enzymes (e.g., ACP desaturases and hycroxylases, ADP-glucose pyrophorylases, ATPases, alcohol dehycrogenases, amylases, amyloglucosidases, catalases, cellulases, cyclooxygenases, decarboxylases, dextrinases, esterases, DNA and RNA polymerases, galactosidases, glucanases, glucose oxidases, GTPases, helicases, hemicellulases, integrases, invertases, isomersases, kinases, lactases, lipases, lipoxygenases, lysozymes, pectinesterases, peroxidases, phosphatases, phospholipases, phophorylases, polygalacturonases, proteinases and peptideases, pullanases, recombinases, reverse transcriptases, topoisomerases, xylanases).

The target agent may serve as a cell-targeting peptide. Thus, the peptide may enable targeting of the anitibody to a target cell, such as a cancer cell. The antibodies conjugated to a cell-targeting peptide in combination with a therapeutic agent and/or imaging agent.

Cell targeting moieties according to the embodiments may be, for example, an antibody, a growth factor, a hormone, a peptide, an aptamer, a small molecule such as a hormone, an imaging agent, or cofactor, or a cytokine. The cell-targeting moiety may target factors in the extracellular environment. For instance, a cell targeting moiety according the embodiments may bind to a liver cancer cell such as a Hep3B cell. It has been demonstrated that the gp240 antigen is expressed in a variety of melanomas but not in normal tissues. Thus, in some embodiments, the compounds of the present disclosure may be used in conjugates with an antibody for a specific antigen that is expressed by a cancer cell but not in normal tissues.

In certain additional embodiments, it is envisioned that cancer cell targeting moieties bind to multiple types of cancer cells. For example, the 8H9 monoclonal antibody and the single chain antibodies derived therefrom bind to a glycoprotein that is expressed on breast cancers, sarcomas and neuroblastomas (Onda et al., 2004). Another example is the cell targeting agents described in U.S. Patent Publication No. 2004/005647 and in Winthrop et al. (2003) that bind to MUC-1, an antigen that is expressed on a variety cancer types. Thus, it will be understood that in certain embodiments, cell targeting peptides according to the embodiments may be targeted against a plurality of cancer or tumor types.

Additionally, certain cell surface molecules are highly expressed in tumor cells, including hormone receptors such as human chorionic gonadotropin receptor and gonadotropin releasing hormone receptor (Nechushtan et al., 1997). Therefore, the corresponding hormones may be used as the cell-specific targeting moieties in cancer therapy. Additionally, the cell targeting moiety that may be used include a cofactor, a sugar, a drug molecule, an imaging agent, or a fluorescent dye. Many cancerous cells are known to over express folate receptors and thus folic acid or other folate derivatives may be used as conjugates to trigger cell-specific interaction between the conjugates of the present disclosure and a cell (Campbell, et al., 1991; Weitman, et al., 1992).

Since a large number of cell surface receptors have been identified in hematopoietic cells of various lineages, ligands or antibodies specific for these receptors may be used as cell-specific targeting moieties. IL-2 may also be used as a cell-specific targeting moiety in a chimeric protein to target IL-2R⁺ cells. Alternatively, other molecules such as B7-1, B7-2 and CD40 may be used to specifically target activated T cells (The Leucocyte Antigen Facts Book, 1993, Barclay, et al. (eds.), Academic Press). Furthermore, B cells express CD19, CD40 and IL-4 receptor and may be targeted by moieties that bind these receptors, such as CD40 ligand, IL-4, IL-5, IL-6 and CD28. The elimination of immune cells such as T cells and B cells is particularly useful in the treatment of lymphoid tumors.

Other cytokines that may be used to target specific cell subsets include the interleukins (IL-1 through IL-15), granulocyte-colony stimulating factor, macrophage-colony stimulating factor, granulocyte-macrophage colony stimulating factor, leukemia inhibitory factor, tumor necrosis factor, transforming growth factor, epidermal growth factor, insulin-like growth factors, and/or fibroblast growth factor (Thompson (ed.), 1994, The Cytokine Handbook, Academic Press, San Diego). In some aspects, the targeting polypeptide is a cytokine that binds to the Fn14 receptor, such as TWEAK (see, e.g., Winkles, 2008; Zhou, et al., 2011 and Burkly, et al., 2007, incorporated herein by reference).

A skilled artisan recognizes that there are a variety of known cytokines, including hematopoietins (four-helix bundles) [such as EPO (erythropoietin), IL-2 (T-cell growth factor), IL-3 (multicolony CSF), IL-4 (BCGF-1, BSF-1), IL-5 (BCGF-2), IL-6 IL-4 (IFN-β2, BSF-2, BCDF), IL-7, IL-8, IL-9, IL-11, IL-13 (P600), G-CSF, IL-15 (T-cell growth factor), GM-CSF (granulocyte macrophage colony stimulating factor), OSM (OM, oncostatin M), and LIF (leukemia inhibitory factor)]; interferons [such as IFN-γ, IFN-α, and IFN-β); immunoglobin superfamily (such as B7.1 (CD80), and B7.2 (B70, CD86)]; TNF family [such as TNF-α (cachectin), TNF-β (lymphotoxin, LT, LT-α, LT-β, CD40 ligand (CD4OL), Fas ligand (FasL), CD27 ligand (CD27L), CD30 ligand (CD3OL), and 4-1BBL)]; and those unassigned to a particular family [such as TGF-β, IL 1α, IL-1β, IL-1 RA, IL-10 (cytokine synthesis inhibitor F), IL-12 (NK cell stimulatory factor), MIF, IL-16, IL-17 (mCTLA-8), and/or IL-18 (IGIF, interferon-γ inducing factor)]. Furthermore, the Fc portion of the heavy chain of an antibody may be used to target Fc receptor-expressing cells such as the use of the Fc portion of an IgE antibody to target mast cells and basophils.

Furthermore, in some aspects, the cell-targeting moiety may be a peptide sequence or a cyclic peptide. Examples, cell- and tissue-targeting peptides that may be used according to the embodiments are provided, for instance, in U.S. Pat. Nos. 6,232,287; 6,528,481; 7,452,964; 7,671,010; 7,781,565; 8,507,445; and 8,450,278, each of which is incorporated herein by reference.

Thus, in some embodiments, cell targeting moieties are antibodies or avimers. Antibodies and avimers can be generated against virtually any cell surface marker thus, providing a method for targeted to delivery of GrB to virtually any cell population of interest. Methods for generating antibodies that may be used as cell targeting moieties are detailed below. Methods for generating avimers that bind to a given cell surface marker are detailed in U.S. Patent Publications Nos. 2006/0234299 and 2006/0223114, each incorporated herein by reference.

### B. Formulation and Administration

The present disclosure provides pharmaceutical compositions comprising antibodies conjugated to target agent(s). Such compositions comprise a prophylactically or therapeutically effective amount of an antibody or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Active vaccines are also envisioned where antibodies like those disclosed are produced in vivo in a subject at risk of Poxvirus infection. Such vaccines can be formulated for parenteral administration, e.g., formulated for injection via the intradermal, intravenous, intramuscular, subcutaneous, or even intraperitoneal routes. Administration by intradermal and intramuscular routes are contemplated. The vaccine could alternatively be administered by a topical route directly to the mucosa, for example by nasal drops, inhalation, or by nebulizer. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### II.Kits

In various aspects of the embodiments, a kit is envisioned containing therapeutic agents and/or other therapeutic and delivery agents. In some embodiments, the present embodiments contemplates a kit for preparing and/or administering an antibody composition of the embodiments. The kit may comprise one or more sealed vials containing any of the pharmaceutical compositions of the present embodiments. The kit may include, for example, conjugated antibodies as well as reagents to prepare, formulate, and/or administer the components of the embodiments or perform one or more steps of the inventive methods. The kit may comprise an expression system for producing the conjugated antibodies, such as the ncAA linker. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

The kit may comprise one or more reagents for a biotechnology product or assay, such as an antibody-HRP conjugate or antibody fluorophore conjugate. The kit may further comprise regents for in vitro assays such as western blots, flow cytometry, immunoprecipitation, ELISA, or immunofluorescence.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### III. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Proximity-induced Site-specific Antibody Conjugation

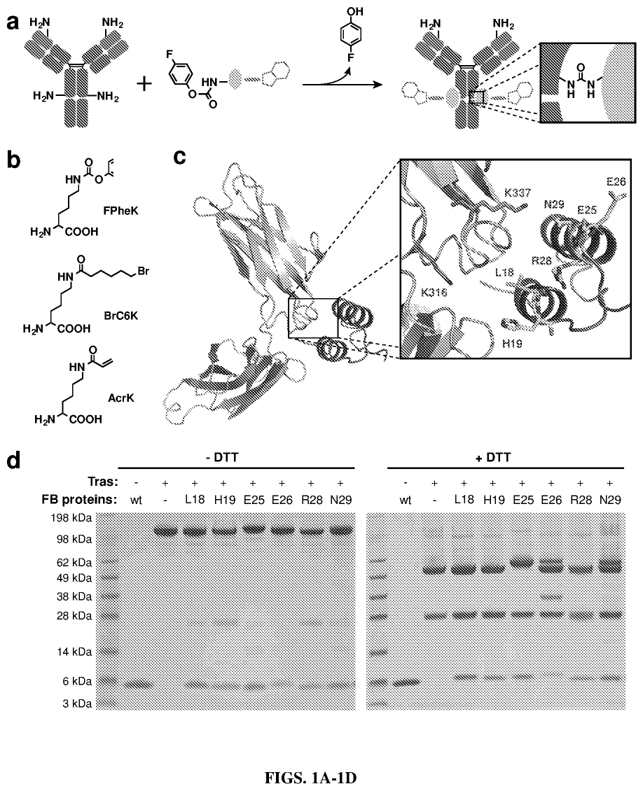

To selectively react with an adjacent native amino acid, such as cysteine or lysine, a number of ncAAs with reactive halide, aryl ketone, Michael acceptor, aryl isothiocyanate, or aryl carbamate side chains were developed^{[13-18]}. These ncAAs were genetically incorporated into various proteins to enhance reactivity between proteins and small molecules or to capture transient protein-protein interactions. Because of the high efficiency and selectivity of these crosslinking reactions, it was envisioned that proximity-enhanced bio-reactivity could be used to site-specifically label antibodies without antibody engineering (FIG. 1A). Specifically, ncAAs allowing crosslinking to a proximal lysine residue were introduced into specific sites of peptides that have well-characterized binding sites at the fragment crystallizable (Fc) or antigen-binding (Fab) fragment of antibodies (FIG. 1B). Upon binding to the antibody, the crosslinking ncAA-containing peptide enable proximity-induced covalent attachment of the crosslinking ncAA to the nearby lysine residue of the antibody. Because of the high labeling selectivity and mild crosslinking conditions, it was reasoned that this method would be able to covalently attach a variety of functional reagents to the large library of existing antibodies.

To avoid the potential disruption of the Fab-binding site and the Fc receptor-binding site, the B domain of protein A (FB protein) from Staphylococcus aureus was used ^{[19]}. The FB protein is a small and stable protein that binds to the C_{H}2-C_{H}3 junction of the immunoglobulin G (IgG) antibody. The co-crystal structure (PDB: 1FC2) reveals that Leu18 and His19 in the FB protein come into close proximity to Lys316 in the human IgG and that FB residues Glu25, Glu26, Arg28, and Asn29 are close to Lys337 in the IgG (FIG. 1C). To test if an electrophilic ncAA-containing FB domain could be used to site-specifically label antibodies, 4-fluorophenyl carbamate lysine (FPheK, FIG. 1B), an ncAA that can react with a proximal lysine to form a stable crosslink, was site-specifically incorporated into the above six residues of the FB protein using the ncAA technology reported previously^{[16]}. All FB mutants containing FPheK were purified by Ni-NTA chromatography and fully characterized by SDS-PAGE and ESI-MS. Trastuzumab (Tras), the native human epidermal growth factor receptor 2 (HER2)-specific antibody, was incubated with 8 equiv of each FB mutant separately in PBS buffer (pH 8.5) at 37° C. for 48 h. Most of the mutants showed no or very weak crosslinking to Tras, with the exception being the variant with FPheK at position 25 (FIG. 1D). Reducing SDS-PAGE analysis of Tras incubated with the FB containing an E25FPheK mutation (FB-E25FPheK) revealed a new band of 57 kDa, consistent with the formation of the FB-heavy chain complex.

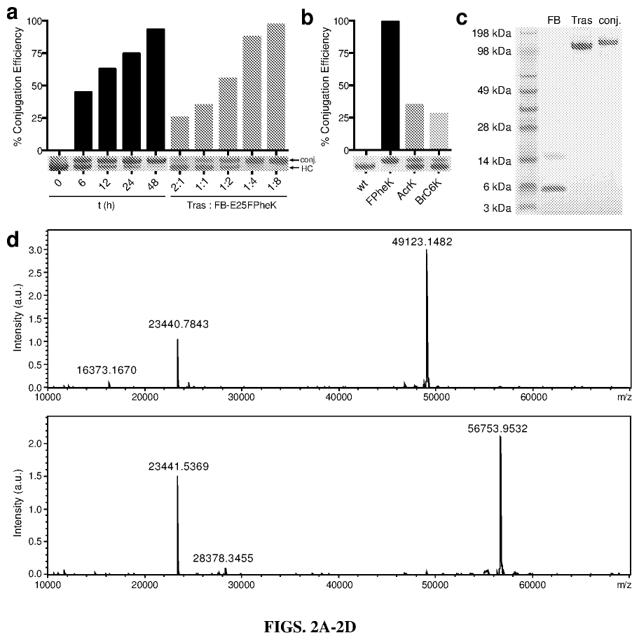

Next, it was sought to optimize the conjugation conditions using Tras and the FB-E25FPheK mutant as model substrates. Based on SDS-PAGE analysis, the modification efficiency improved with both longer reaction time and an increased amount of the FB mutant (FIG. 2A). The conjugation reaction with 8 equiv of FB-E25FPheK mutant for 48 h afforded the Tras-FB conjugate at greater than 95% conjugation yield. Upon reaction completion, the resulting conjugate was washed in an Amicon 50,000 molecular-weight-cutoff protein concentrator to remove excess FB proteins. Analysis by ESI-MS of the Tras-FB conjugate revealed a mass difference of 7631 Da between the unconjugated (49,123 Da) and FB-conjugated heavy chain (56,754 Da, FIG. 2D). Unreacted IgG-FB heavy chain or degradation products were undetectable by SDS-PAGE or ESI-MS (FIG. 2C and D).

To explore the efficiencies of various electrophilic moieties, two other lysine analogs, N-acryloyl-lysine (AcrK) and 2-amino-6-(6-bromohexanamido)hexanoic acid (BrC6K), were synthesized and incorporated into the Glu25 residue that exhibited the best crosslinking efficiency (FIG. 1B)^{[15,18]}. The substitution of Glu25 with AcrK or BrC6K showed 20-40% conjugation efficiency, which was significantly lower than that of FPheK (95%) (FIG. 2B). Thus, FPheK was used in the latter conjugation experiment.

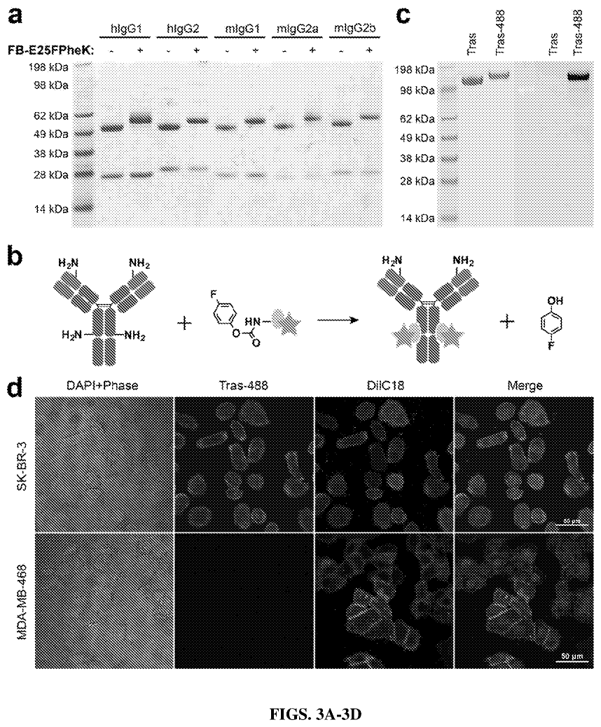

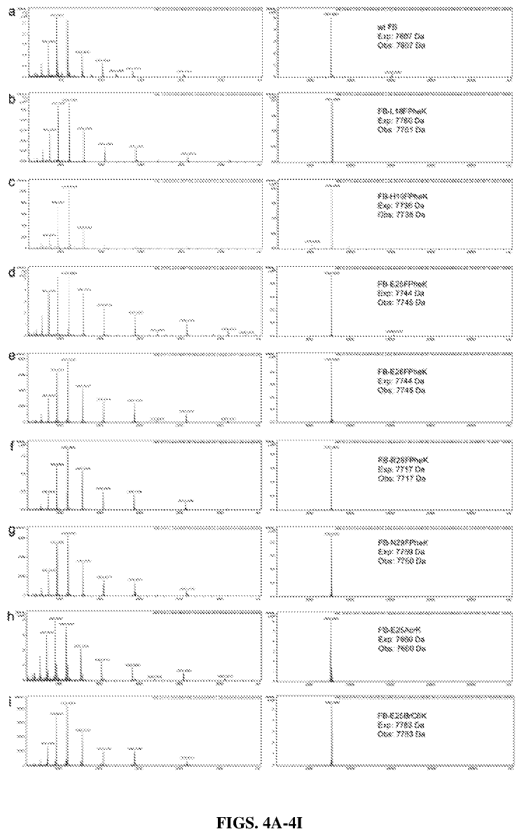

Antibodies, particularly of the IgG isotype, are widely used as affinity reagents in many research applications, disease diagnostics, and therapies. To demonstrate the generality of the conjugation method developed above, the conjugation efficiency and specificity of IgGs from different species and subclasses were tested. The FB-E25FPheK protein was crosslinked to human IgG1 and IgG2 and mouse IgG1, IgG2a, and IgG2b with efficiencies of 96%, 99%, 99%, 91%, and 99%, respectively (FIG. 3A). Human IgG1 is the predominant antibody subclass used today for antibody therapy, whereas mouse IgG1 is the most employed antibody subclass for biotechnological applications ^{[20]}. The successful site-specific labeling of these antibody subclasses suggests a high potential for application of the developed proximity-induced antibody conjugation method.

Fluorophore-labeled monoclonal antibodies provide a powerful tool for disease detection, intraoperative imaging, and pharmacokinetic characterization of therapeutic reagents. Attaching a fluorophore to the mutant FB protein should allow for site-specific introduction of an imaging reagent to antibodies without antibody engineering. To explore this possibility, the FB-E25FPheK protein was functionalized with an Alexa Fluor™ 488 succinimidyl ester by nonspecific conjugation to lysine residues (FIG. 3B). After an overnight reaction, the resulting conjugate was buffer-exchanged into pH 8.5 PBS buffer and added to the native trastuzumab antibody. The resulting Alexa Fluor™ 488-labeled trastuzumab (Tras-488) was further purified by a protein-L column (FIG. 3C). The conjugation reaction afforded Tras-488 in greater than 98% conjugation yield, as observed by SDS-PAGE analysis (FIG. 3C). A band was observed only for the Tras-488 under UV transillumination, indicating the successful incorporation of the fluorophore (FIG. 3C). With the fluorophore-labeled Tras in hand, its utility was tested for visualizing antigens on the breast cancer cell surfaces. HER2-positive SK-BR-3 cells and HER2-negative MDA-MB-468 cells were treated for 30 min with Tras-488 prepared above. Confocal fluorescent imaging indicated that cell-surface-associated fluorescence was exhibited only by SK-BR-3 cells, while HER2-negative MDA-MB-468 cells did not exhibit any associated fluorescence (FIG. 3D). These results indicated that antibodies site-specifically modified with a functionalized FB-E25FPheK protein retain their antigen-binding ability.

In conclusion, a novel proximity-induced antibody conjugation platform was developed for the preparation of site-specific antibody conjugate without the need for antibody engineering. With the introduction of a crosslinking ncAA allowing covalent bond formation with a proximal lysine, affinity peptides with various functional moieties can be site-specifically conjugated to antibodies. This platform enables rapid, site-specific, efficient conjugation to the existing native antibody and further facilitate antibody conjugate discovery and design.

### Example 2—Materials and Methods

LB agar and LB broth were ordered from BD Difco™. Isopropyl-β-D-thiogalactoside (IPTG) was purchased from Anatrace. SeeBlue™ Pre-stained Protein Standard and 4-12% Bis-Tris gels for SDSPAGE were purchased from Invitrogen. QuickChange Lightning Multi Site-Directed Mutagenesis Kit was purchased from Agilent Technologies. Oligonucleotide primers were purchased from Integrated DNA Technologies and Eurofins Genomics (Table 1). Plasmid DNA preparation was carried out with the GenCatch™ Plus Plasmid DNA Miniprep Kit and GenCatch™ Advanced Gel Extraction Kit. BugBuster™ Protein Extraction Reagent was purchased from Novagen, Protease inhibitor Cocktail was purchased from biotool, Pierce™ Universal Nuclease was purchased from Thermo Scientific. Ni-NTA Agarase was purchased from QIAGBN. Unless otherwise mentioned, all solvents and chemicals for synthesis were purchased from Alfa Aesar and Fisher Chemical and used as received without further purification, unless otherwise specified. Alexa Fluor™ 488 succinimidyl ester (Cat No: A20000) and Hoechst 33342 (Cat No: H1399) were purchased from Life Technologies™. 3,3-Dioctadecyloxacarbocyanine perchlorate (DiIC18, Cat No: M1197) was purchased from Marker Gene Technologies, Inc. Human IgG2 (Cat No: BE0301), Mouse IgG1 (Cat No: BE0083), Mouse IgG2a (Cat No: BE0085) and Mouse IgG2b (Cat No: BE0086) isotype control were purchased from BioCell.

Confocal fluorescent images of living cells were obtained using Nikon A1R-si Laser Scanning Confocal Microscope (Japan), equipped with lasers of 405/488/561/638 nm.

Synthesis of 4-fluorophenyl carbamate lysine (FPheK).

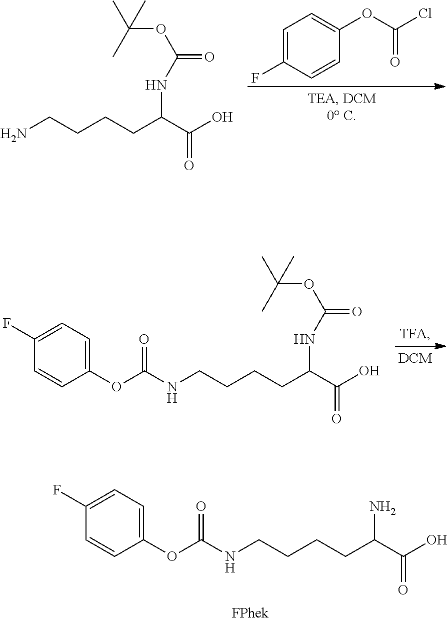 [SMILES: CC(C)(C)OC(=O)NC(CCCCN)C(=O)O ; O=C(Cl)Oc1ccc(F)cc1 ; CC(C)(C)OC(=O)NC(CCCCNC(=O)Oc1ccc(F)cc1)C(=O)O ; NC(CCCCNC(=O)Oc1ccc(F)cc1)C(=O)O]

Nα-Boc-L-lysine (4 g, 16.2 mmol) was dissolved in 50 mL anhydrous DCM and then TEA (5.6 mL, 2.5 equiv.) was added at 0° C. After stirring for 10 min, 4-fluorophenyl chloroformate (2.2 mL, 1.05 equiv.) was added dropwise, and the mixture was stirred at room temperature under a nitrogen atmosphere overnight. The mixture was poured into 50 mL H₂O, and the pH was adjusted to 3 with 2 M aq. AcOH. The organic phase was separated and dried with Na2SO4. The solvent was evaporated, and the intermediate was purified by flash column chromatography on silica (DCM:MeOH, 10:1) and obtained as colorless oil. 40 mL fresh DCM was used to dissolve the intermediate followed by addition of 10 mL TFA. The solution was stirred at room temperature for 5 h. The solvent was evaporated. The crude product was dissolved in methanol and precipitated in Et₂O. the product was dried under vacuum and obtained as light yellow powder (2.2 g, 34%). 1H-NMR (400 MHz, Methanol-D4): δ7.08-7.10(m, 4H), 3.93 (t, J=6.4 Hz, 1H), 3.27-3.24 (m, 2H), 1.89-1.81 (m, 2H), 1.70-1.60(m, 2 H), 1.52-1.48 (m, 2H). 13C-NMR (400 MHz, Methanol-D4): δ172.06, 170.41, 161.45, 157.53, 148.65, 124.55, 117.01, 54.62, 41.54, 30.44, 28.64, 23.53. ESI-MS: m/z calcd 285.1251[M+H]⁺, found 285.1374.

Synthesis of N-acryloyl-lysine (AcrK).

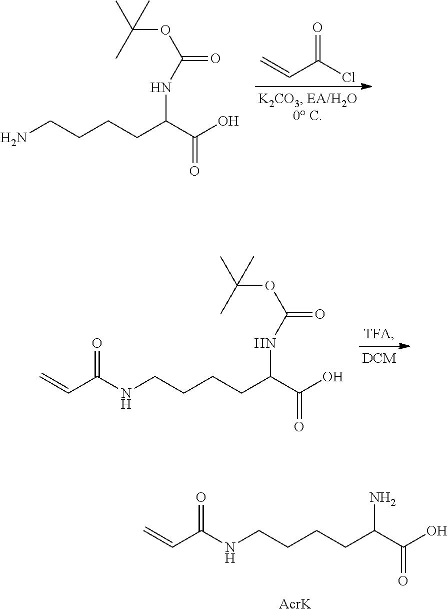

Nα-Boc-L-lysine (2.46 g, 10.0 mmol) and Na₂CO₃ (2.12 g, 20.0 mmol) was dissolved in 200 mL ethyl acetate/H₂O (1:1) at 0° C. Acryloyl chloride (0.9 g, 11.0 mmol) was added dropwise over 10 min. The solution was stirred at room temperature under a nitrogen atmosphere overnight. 2 M aq. AcOH was then added to adjust the pH to 3. The mixture was extracted with ethyl acetate (200 mL, 3 times). The organic phase was separated and dried with Na₂SO₄. 20 mL fresh DCM was used to dissolve the intermediate followed by addition of 5 mL TFA. The solution was stirred at room temperature for 5 h. The solvent was evaporated. The crude product was dissolved in methanol and precipitated in Et₂O. The product was dried under vacuum and obtained as white solid in 80% yield (1.8 g). 1H NMR (600 MHz, D2O) δ=1.38-1.47 (m, 2 H), 1.55-1.59 (m, 2 H), 1.89-1.97 (m, 2 H), 3.26 (dd, J1=6.94 Hz, J2 =13.73, 2 H), 4.03 (dd, J1=6.21 Hz, J2=12.47 Hz, 2 H), 5.71 (d, J=10.30 Hz, 1 H), 6.12-6.24 (m, 2 H). ESI-MS: m/z calcd 201.1239 [M+H]⁺, found 201.1259.

Synthesis of 2-amino-6-(6-bromohexanamido)hexanoic acid (BrC6K).

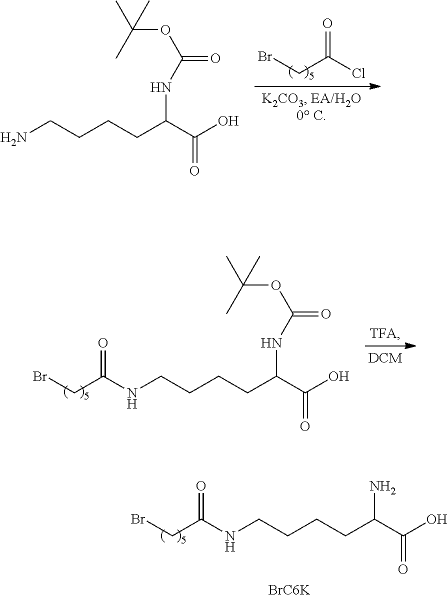

Nα-Boc-L-lysine (2.46g, 10.0 mmol) was dissolved in 200 ml mixture solvent of THF/DCM (1:1) and DIEA (12.0 mmol, 2.1 mL) at 0° C.; 6-bromohexanoyl chloride (13 mmol, 2.0 mL) was added. The solution was stirred at room temperature under a nitrogen atmosphere overnight. 2 M aq. AcOH was then added to adjust the pH to 3. The mixture was extracted with ethyl acetate (200 mL, 3 times). The organic phase was separated and dried with Na₂SO₄. The crude material was purified by flash silica gel chromatography using eluent solvent with DCM:MeOH (10:1). The product was isolated as a yellow oil (2.8 g, 67%). The pure product was dissolved in dichloromethane (15 mL) followed by addition of 5 mL TFA. The reaction mixture was stirred for 5 h. After the reaction completed, the solvent was concentrated under reduced pressure. The residue was dissolved in methanol and precipitated in Et2O. The white solid was washed with Et2O to give the final product. (1.7 g, 80%). BrC6K (4), 1H NMR (700 MHz, DMSO-D6): 3.88 (t, J=5.6 Hz, 1H), 3.51 (t, J=7.0 Hz, 2H), 3.01 (t, J=7.0 Hz, 2H), 2.04 (t, J=6.3 Hz, 2H), 1.30-1.79 (m, 12H). ESI-MS: m/z calcd 323.0970 [M+H]⁺, found 323.0993.

Plasmid construction: The FPheKRS gene was generated by PCR using primers CY012 and CY013, and inserted into the pUltra-MbPy1RS plasmid using restriction enzyme Notl provide pUltra-FPheKRS. The plasmids to express FB mutants were generated by site-directed mutagenesis using primers CY031, CY038, CY039, CY040, CY041 and CY042, using pET22b-T5-FB as the template with QuickChange Lightning Multi Site-Directed Mutagenesis Kit (Agilent Technologies).

Expression and purification of FB protein: The pET22b-T5-FB-E25TAG and pUltra-FPheKRS plasmids were co-transformed into E. coli DH10B strains. Cells were grown in LB media, supplemented with ampicillin (50 ug/mL), spectinomycin (25 ug/mL) and 1 mM FPheK at 37° C. When the OD reached to 0.6, 1 mM IPTG was added to the culture, and the culture was grown over night at 30° C. The cells were harvested by centrifugation at 4,700×g for 10 min and the proteins were purified on Ni-NTA resin following the manufacture's (Qiagen) instructions.

Site-specific antibody-FB protein conjugation: FB protein mutants were expressed and purified as described above. Antibody (50 μM) was co-incubated with eight equiv of FB mutants in pH 8.5 PBS buffer for 2 days. The resulting antibody conjugates were purified by protein-L column following the manufacture's (GE Healthcare Life Sciences) instructions. The conjugation efficiency was analyzed using ImageQuant TL.

Preparation of Alexa Fluor™488-labeled trastuzumab conjugate: FB-E25FPheK protein (20 uL, 2.3 mg/mL in DPBS buffer with Ca²⁺ and Mg²⁺, pH 8.5) was reacted with 10 equivalents of Alexa Fluor™ 488 carboxylic acid, succinimidyl ester at 37° C. for 12 hours. The resulting FITC-labeled FB protein was then purified by Ni-NTA chromatography following the manufacture's (Qiagen) instructions and buffer-exchanged to PBS buffer (pH 8.5) using an Amicon 3,000 molecular-weight-cutoff concentrator. It was then reacted with trastuzumab (10 uL, 50 uM) at 37° C. for 2 days. The resulting conjugate was then purified by Protein-L column following the manufacture's (GE Healthcare Life Sciences) instructions, followed by adjusting pH to 7.0. The isolated protein was characterized by SDS-PAGE analysis followed by Coomassie staining Protein concentration was measured using Coomassie Plus (Bradford) Protein Assay kit from Pierce.

Fluorescence Microscopy: Confocal fluorescent imaging of living cells was performed using Nikon A1R-si Laser Scanning Confocal Microscope (Japan), equipped with lasers of 405/488/561/638 nm. DiIC18 and Hoechst 33342 were prepared as 2 mM DMSO stock solution and 10 mg/mL water solution, respectively. The stock solution was diluted to the working concentration in complete medium (10 μM and 10 μg/mL, respectively). SK-BR-3 cells and MDA-MB-468 cells were incubated in complete medium (RPMI 1640 Medium or Dulbecco' s modified Eagle's Medium, respectively, supplemented with 10% fetal bovine serum (FBS) and 1% penicillin-streptomycin) at 37° C. in atmosphere containing 5% CO₂.

SK-BR-3 cells and MDA-MB-468 cells were grown to about 80% confluency in 8-well confocal imaging chamber plates. Cells were incubated with 30 nM Tras-488 for 30 min and then fixed by 4% paraformaldehyde for 15 min Cells were washed with PBS (pH 7.4) for three times. Then Cells were incubated with DiIC18(3) for 20 min and Hoechst 33342 for 5 min, respectively. After washed with PBS (pH 7.4) for three times, confocal imaging was carried out. Images were taken under conditions as follows: 60× immersion lens with a resolution of 1024×1024 and a speed of 0.25 frame per second; 30% laser power for Hoechst 33342, 405 nm excitation wavelength and 425 to 475 nm detector slit; 50% laser power for Tras-488, 488 nm excitation wavelength and 500 to 530 nm detector slit; 15% laser power for DiIC18(3), 561 nm excitation wavelength and 582 to 617 nm detector slit. Differential interference contrast (DIC) and fluorescent images were processed and analyzed using ImageJ.

TABLE 1
DNA sequences.

| oligo- | | |
|---|---|---|
| nucleotide | Description | Sequence (5′-3′) |
| CY039 | L18TAG | GGAGCAACAGAACGCCTTTTATGA |
| | | AATCTAGCATTTGCCAAACCTTAA |
| | | CGAGGAACAGAG |
| | | (SEQ ID NO: 4) |
| CY040 | H19TAG | GCAACAGAACGCCTTTTATGAAAT |
| | | CCTTTAGTTGCCAAACCTTAACGA |
| | | GGAACAGAGGAA |
| | | (SEQ ID NO: 5) |
| CY031 | E25TAG | TGAAATCCTTCATTTGCCAAACC |
| | | TTAACTAGGAACAGAGGAATGCG |
| | | TTTATTCAATCACT |
| | | (SEQ ID NO: 6) |
| CY041 | E26TAG | AATCCTTCATTTGCCAAACCTTA |
| | | ACGAGTAGCAGAGGAATGCGTTT |
| | | ATTCAATCACTTAA |
| | | (SEQ ID NO: 7) |
| CY042 | R28TAG | TCATTTGCCAAACCTTAACGAGG |
| | | AACAGTAGAATGCGTTTATTCAA |
| | | TCACTTAAGGATGA |
| | | (SEQ ID NO: 8) |
| CY038 | N29TAG | TTTGCCAAACCTTAACGAGGAAC |
| | | AGAGGTAGGCGTTTATTCAATCA |
| | | CTTAAGGATGATCC |
| | | (SEQ ID NO: 9) |
| CY012 | FPheKRS-f | TCACAaAGGAggtGCGGCCGCAT |
| | | GGATAAAAAACCATTAGATGTTT |
| | | TAATATCTGCGACC |
| | | (SEQ ID NO: 10) |
| CY013 | FPheKRS-r | gaccgtttaaacGCGGCCGCTTA |
| | | TAGATTGGTTGAAATCCCATTAT |
| | | AGTAAGATTCGGAC |
| | | (SEQ ID NO: 11) |

DNA sequence of WT-FB protein:
(SEQ ID NO: 3)
ATGGTGGACAATAAATTCAACAAGGAGCAACAGAACGCCTTTTATGAAAT
CCTTCATTTGCCAAACCTTAACGAGGAACAGAGGAATGCGTTTATTCAAT
CACTTAAGGATGATCCAAGTCAATCGGCCAACCTGTTGGCGGAGGCCAAA
AAATTAAATGACGCACAAGCGCCTAAAGGATCCCACCACCATCATCATCA
TTGA

### Example 3—Proximity-induced Site-specific Antibody Conjugation with Solid-Phase Peptide Synthesis

Proteins can be biosynthesized in the cells with the information in genes. With cognate tRNA synthetase, natural amino acids can be amino-acylated to the tRNA in response with the codons. However, as the transcription process is under strict bio-orthogonal pathway, without genetic code expansion, the cells cannot recognize noncanonical amino acids and incorporate them into proteins. As an alternative, solid-phase synthesis has been developed and widely used for peptide synthesis. In solid-phase synthesis, peptide was synthesized from carboxylic acid (C-terminal) to amino group (N-terminal). Starting from a solid material (polystyrene beads), amino acids with protection groups can be stepwise assembled together to make a long chain peptide. The technique of peptide synthesis has improved a lot since it was developed in 1963 by Merrifield. Till now, Fmoc-based peptide synthesis is the most common strategy as it can be utilized under mild reaction conditions. An additional advantage of solid-phase peptide synthesis is that technically it's able to incorporate any chemicals with the reactive amine and carboxylic acid groups into peptide sequence thus it facilitates the peptide modification for biological and pharmaceutical applications.

The FPheK-labeled FB peptide from Example 1 was prepared using genetic code expansion technology (FIG. 5). However, in order to explore the applications of ‘pClick’ antibody conjugation method of Example 1, a truncated FB (amino acid sequence: FNKEQQNAFYEILHLPNLNXEQRNAFIQSLKDD; SEQ ID NO: 2 (X=MMT-Lys)) (ssFB) which remains the function of antibody binding was synthesized via Fmoc-based solid-phase peptide synthesis. As shown in FIG. 8, starting from rink amide resin, ssFB with N-terminal acetylation was stepwise synthesized with all the amino acid side chains protected. After finishing the whole peptide, the MMT (monomethoxytrityl) protection group was selectively removed with 10% AcOH solution (AcOH:TFEDCM=1:2:7). The exposing free amine was then reacted with 4-fluorophenyl chloroformate under mild basic conditions for FPheK formation.

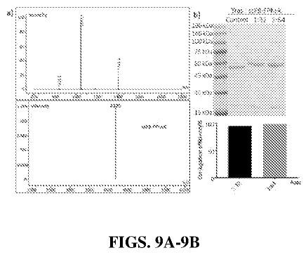

TFA and scavengers were used to cleave the peptide from the resin, remove and quench all protections at the same time. After that, the peptide was precipitated by ice-cold ether and further purified with HPLC and characterized by ESI-MS (FIG. 9A). To functionalize the peptide, it was first lyophilized and totally denatured with 8 M urea solution. A stepwise dialysis protocol was used to slowly remove urea and refold the peptide. 32 equiv of ssFB peptide was then mixed with Tras for 2 days at 37° C. in PBS (pH 8.5) buffer. Reducing SDS-PAGE analysis (FIG. 9B) revealed a clear band shift of Tras heavy chain which confirmed the Tras-ssFB conjugate formation with over 95% yield.

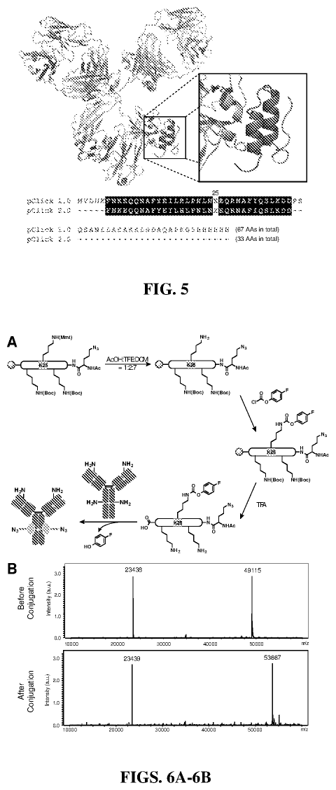

On the basis of the crystal structure of the antibody:FB complex, a 33 AA affinity peptide was generated containing two helixes involved in peptide binding to the antibody (FIG. 5). To enable selective introduction of FPheK at position-25 of the truncated FB peptide, a lysine with an orthogonal protected group, 4-methoxytrityl (Mmt), was first introduced at residue 25, while the other lysine residues were protected with tert-butyloxycarbonyl (Boc) groups (FIG. 6A). The resulting full-length protected peptide was then coupled to an azido-lysine at the N-terminus, followed by orthogonal deprotection of residue Lys25. After site-specific on-resin deprotection, the free Lys25 was reacted with 4-fluorophenyl chloroformate to yield FPheK at residue 25, followed by resin washing to remove excess 4-fluorophenyl chloroformate. Trifluoroacetic acid was then used to free the peptide from the resin to yield the final azide-labeled FB containing the E25FPheK mutation (AzFB). The truncated azide-labeled FB peptide was prepared with good purity and yield of about a gram.

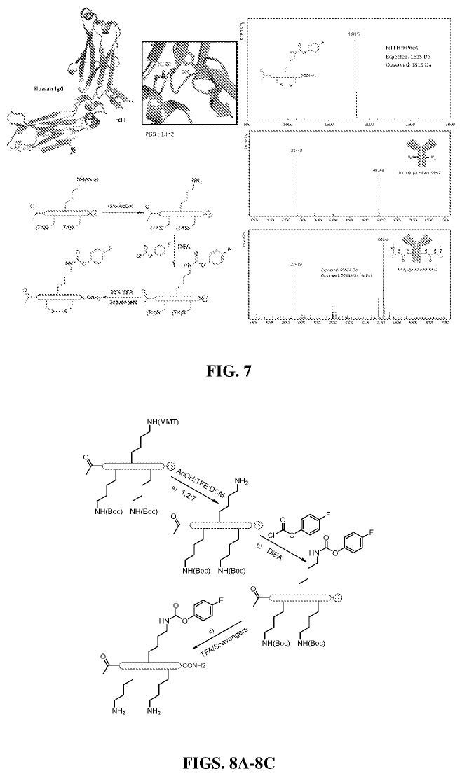

The antibody conjugation efficiency of this short FB peptide mutant was examined by incubating Tras with 16 equivalents of AzFB for 48 h to yield Tras with an azide functional moiety (Tras-azide-FB). Reducing SDS-PAGE analysis revealed a clear band shift of the Tras heavy chain, confirming greater than 95% formation of the Tras-azide-FB conjugate. ESI-MS spectrometry analysis revealed a 4551 Da peak shift between unconjugated Tras heavy chain (49114 Da) and Tras-azide-FB heavy chain, in agreement with the mass of one AzFB peptide (FIG. 7B). Furthermore, used the strain-promoted alkyne-azide cycloaddition (SPAAC) reaction was used to demonstrate that the azide-labeled Tras is reactive with various bicyclo[6.1.0]nonyne (BCN) reagents.

Further, Cyclic Fclll-FPheK peptide (FIG. 7) was synthesized by following the protocol of Fmoc-based peptide synthesis. DDCAWXLGELVWCT (X=MMT-Lys) (SEQ ID NO:12). To start with, Rink amide resin with a Fmoc protected amine was used as the solid support. 25% piperidine in DMF was first used to remove Fmoc group. After five times DMF wash, the free amine could be checked by chloranil test. Pre-activated HATU/Fmoc-amino acid mixture in DMF was then added into the reaction vessel for amide bond formation followed by another chloranil test. The next amino acid was coupled to the peptide via the same reaction cycle. Once the peptide synthesis was completed, the N-terminal of the peptide was acetylated by acetic anhydride.

In order to incorporate FPheK into the peptide, the MMT group was first selectively removed by 10% acetic acid (AcOH:TFE:DCM=1:2:7). After DMF/DCM wash and chloranil test, 2 equiv of 4-fluorophenyl chloroformate and 4 equiv of DiEA were added into the reaction vessel for FPheK formation.

Once the reaction was completed, proper amount of TFA and scavengers (water, anisole, triisopropyl silane, EDT) were added into the vessel to cleave the peptide from the resin, remove and quench all other protection groups. The peptide was then precipitated by cold ether, purified by HPLC and lyophilized.

Before usage, the Fclll-FPheK peptide was dissolved in PBS (pH=8.5)/DMF solution and open to air for disulfide bond formation. After that, 32 equiv of cyclic Fclll-FPheK peptides were co-incubated with Tras antibody at 37° C. for 2 days and purified by 100,000 Da concentrator. The successful conjugation reaction was confirmed by ESI-MS analysis.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

[1] T. Kline, A. R. Steiner, K. Penta, A. K. Sato, T. J. Hallam, G. Yin, Pharm Res 2015, 32, 3480-3493.

[2] L. Wang, G. Amphlett, W. A. Blattler, J. M. Lambert, W. Zhang, Protein Sci. 2005, 14, 2436-2446.

M. M. C. Sun, K. S. Beam, C. G. Cerveny, K. J. Hamblett, R. S. Blackmore, M. Y. Torgov, F. G. M. Handley, N. C. Ihle, P. D. Senter, S. C. Alley, Bioconjug. Chem. 2005, 16, 1282-1290.

[4] P. Agarwal, C. R. Bertozzi, Bioconjugate Chem. 2015, 26, 176-192.

[5] J. R. Junutula, H. Raab, S. Clark, S. Bhakta, D. D. Leipold, S. Weir, Y. Chen, M. Simpson, S. P. Tsai, M. S. Dennis, et al., Nature Biotechnology 2008, 26, 925-932.

[6] P. Agarwal, J. van der Weijden, E. M. Sletten, D. Rabuka, C. R. Bertozzi, Proc. Natl. Acad. Sci. U.S.A. 2013, 110, 46-51.

[7] I. S. Carrico, B. L. Carlson, C. R. Bertozzi, Nature Chemical Biology 2007, 3, 321-322.

[8] J. E. Hudak, R. M. Barfield, G. W. de Hart, P. Grob, E. Nogales, C. R. Bertozzi, D. Rabuka, Angew. Chem. Int. Ed. Engl. 2012, 51, 4161-4165.

[9] B Ramakrishnan, P. K. Qasba, J. Biol. Chem. 2002, 277, 20833-20839.

[10] E. Boeggeman, B. Ramakrishnan, M. Pasek, M. Manzoni, A. Puri, K. H. Loomis, T. J. Waybright, P. K. Qasba, Bioconjug. Chem. 2009, 20, 1228-1236.

[11] J. Y. Axup, K. M. Bajjuri, M. Ritland, B. M. Hutchins, C. H. Kim, S. A. Kazane, R. Halder, J. S. Forsyth, A. F. Santidrian, K. Stafin, et al., PNAS 2012, 109, 16101-16106.

[12] H. Xiao, A. Chatterjee, S. Choi, K. M. Bajjuri, S. C. Sinha, P. G. Schultz, Angew. Chem. Int. Ed. 2013, 52, 14080-14083.

[13] Z. Xiang, H. Ren, Y. S. Hu, I. Coin, J. Wei, H. Cang, L. Wang, Nature Methods 2013, 10, 885-888.

[14] Z. Xiang, V. K. Lacey, H. Ren, J. Xu, D. J. Burban, P. A. Jennings, L. Wang, Angew. Chem. Int. Ed. Engl. 2014, 53, 2190-2193.

[15] X.-H. Chen, Z. Xiang, Y. S. Hu, V. K. Lacey, H. Cang, L. Wang, ACS Chem. Biol. 2014, 9, 1956-1961.

[16] W. Xuan, S. Shao, P. G. Schultz, Angew. Chem. Int. Ed. Engl. 2017, 56, 5096-5100.

[17] W. Xuan, J. Li, X. Luo, P. G. Schultz, Angew. Chem. Int. Ed. Engl. 2016, 55, 10065-10068.

[18] J. L. Furman, M. Kang, S. Choi, Y. Cao, E. D. Wold, S. B. Sun, V. V. Smider, P. G. Schultz, C. H. Kim, J Am. Chem. Soc. 2014, 136, 8411-8417.

[19] J. Deisenhofer, Biochemistry 1981, 20, 2361-2370.

[20] R. Jefferis, Expert Opin Biol Ther 2007, 7, 1401-1413.

What is claimed is:

1. A method for proximity-induced site-specific conjugation of a target agent to an antibody comprising:
(a) providing an affinity compound having a proximity-reactive motif, wherein the affinity compound is conjugated to the target agent; and
(b) bringing the affinity compound into proximity of the antibody for a sufficient period of time to covalently link the affinity compound to said antibody.

2. The method of claim 1, wherein the affinity compound is a small molecule, DNA, RNA, peptide, or protein.

3. The method of claim 1, wherein the affinity compound is an affinity peptide.

4. The method of claim 3, wherein obtaining the affinity peptide is produced by solid-phase synthesis or recombinant expression.

5. The method of claim 3, wherein the solid-phase synthesis is further defined as Fmoc-based solid-phase synthesis.

6. The method of claim 1, wherein the affinity compound is further defined as an antibody-binding compound.

7. The method of claim 1, wherein the proximity-reactive motif comprises a non-canonical amino acid (ncAA).

8. The method of claim 7, wherein the ncAA has the ability to crosslink with an amino acid residue of said antibody.

9. The method of claim 8, wherein said amino acid residue is histidine, serine, threonine, tryptophan, tyrosine, lysine or cysteine.

10. The method of claim 8, wherein said amino acid residue is lysine.

11. The method of claim 7, wherein the ncAA has a reactive halide, fluorosulfate, sulfonyl fluoride, aryl ketone, Michael acceptor, aryl isothiocyanate, or aryl carbamate side chain.

12. The method of claim 7, wherein the ncAA has a carbamate side chain.

13. The method of claim 12, wherein the ncAA is 4-fluorophenyl carbamate lysine (FPheK), phenyl carbamate lysine (PheK), N-acryloyl-lysine (AcrK), or 2-amino-6-(6-bromohexanamido)hexanoic acid (BrC6K), fluorosulfate-L-tyrosine (FSY), 2-amino-3-(4-(3-bromopropoxy)phenyl)propanoic acid (BprY), sulfonyl fluoride phenylalanine, or N-fluoroacetyllysine (FAcK).

14. The method of claim 12, wherein the ncAA is FPheK.

15. The method of claim 1, wherein the affinity compound exhibits binding for the fragment crystallizable (Fc) region, an antigen-binding (Fab) region, or hinge region of said antibody.

16. The method of claim 1, wherein the affinity compound exhibits binding for the CH2 or CH3 region of said antibody.

17. The method of claim 1, wherein the affinity compound exhibits binding for the CH2-CH3 junction of said antibody.

18. The method of claim 1, wherein the affinity compound is a peptide derived from protein A or protein G.

19. The method of claim 18, wherein the peptide derived from protein A is the Z domain or a fragment thereof.

20. The method of claim 1, wherein the affinity compound is an antibody-binding peptide evolved via phage display.

21. The method of clam 17.3, wherein the antibody-binding peptide is FcIII or a fragment thereof.

22. The method of claim 1, wherein the affinity compound is the B domain of protein A (FB protein) from Staphylococcus aureus or a fragment thereof.

23. The method of claim 22, wherein the ncAA is inserted at residue 25 of the FB protein.

24. The method of claim 22, wherein FPheK is inserted at residue 25 of the FB protein (FB-E25FPheK).

25. The method of claim 22, wherein the affinity compound is a fragment of the FB protein.

26. The method of claim 25, wherein the fragment of the FB protein is a peptide of less than 35 amino acids.

27. The method of claim 25, wherein the fragment of the FB protein is a peptide of 33 amino acids in length.

28. The method of claim 27, wherein the peptide comprises SEQ ID NO: 2.

29. The method of claim 1, wherein the affinity compound is FcIII or a fragment thereof.

30. The method of claim 30, wherein the affinity compound is a cyclic FcIII peptide of SEQ ID NO:3.

31. The method of claim 1, wherein the antibody is an IgG, IgM, IgA, IgE, or antigen binding fragment thereof.

32. The method of claim 1, wherein the antibody is a Fab′, a F(ab′)2, a F(ab′)3, a monovalent scFv, a bivalent scFv, a single domain antibody, or nanobody.

33. The method of claim 1, wherein the antibody is a human antibody.

34. The method of claim 1, wherein the antibody is trastuzumab.

35. The method of claim 1, wherein the covalent linking has an efficiency of at least 40%.

36. The method of claim 1, wherein the covalent linking has an efficiency of at least 90%.

37. The method of claim 1, wherein the covalent linking has an efficiency of at least 95%.

38. The method of claim 1, wherein the covalent linking has an efficiency of at least 99%.

39. The method of claim 1, wherein the target agent is an imaging agent and/or therapeutic agent.

40. The method of claim 39, wherein the therapeutic agent is a toxin or chemotherapeutic agent.

41. The method of claim 39, wherein the imaging agent is a fluorophore or radionuclide.

42. The method of claim 39, wherein the imaging agent is a PET probe or MRI probe.

43. The method of claim 1, wherein the target agent is a drug, small molecule, DNA, RNA, small molecule, protein, peptide, enzyme, nanoparticle, virus, cell, saccharide, antibody or fragment thereof.

44. The method of claim 43, wherein the antibody is an Fc, Fab, scFv, single-domain antibody, or κ-light chain.

45. The method of claim 1, wherein more than one target agent is conjugated to said antibody.

46. The method of claim 45, wherein 2, 3, 4, or 5 target agents are conjugated to said antibody.

47. The method of claim 46, wherein the target agents are conjugated to the antibody at different sites.

48. The method of claim 1, wherein step (b) does not comprise enzymatic treatment.

49. The method of claim 1, wherein the covalent linking of the affinity peptide occurs without the use of other agents or the application of additional treatments.

50. A composition comprising an ncAA linker conjugated to target agent.

51. The composition of claim 50, further comprising an antibody.

52. The composition of claim 51, wherein the composition is produced according to any of claims 1-49.

53. The composition of claim 50, wherein the ncAA linker is covalently attached to the antibody.

54. The composition of claim 50, wherein the ncAA linker comprises an affinity compound having a ncAA.

55. The composition of claim 54, wherein the affinity compound is a small molecule, DNA, RNA, peptide, or protein.

56. The composition of claim 54, wherein the affinity compound is an affinity peptide.

57. The composition of claim 54, wherein the affinity peptide comprises SEQ ID NO: 2.

58. The composition of claim 54, wherein the affinity peptide consists of SEQ ID NO: 2.

59. The composition of claim 56, wherein the affinity peptide is further defined as an antibody-binding peptide.

60. The composition of claim 50, wherein the target agent is an imaging agent and/or therapeutic agent.

61. The composition of claim 60, wherein the therapeutic agent is a toxin.

62. The composition of claim 60, wherein the imaging agent is a fluorophore or radionuclide.

63. The composition of claim 60, wherein the therapeutic agent is a chemotherapeutic agent.

64. The composition of claim 50, wherein the target agent is a drug, DNA, RNA, small molecule, protein, peptide, enzyme, nanoparticle, virus, cell, saccharide, antibody or fragment thereof.

65. The composition of claim 50, wherein the ncAA has the ability to crosslink with an amino acid residue of said antibody.

66. The composition of claim 65, wherein said amino acid residue is lysine or cysteine.

67. The composition of claim 50, wherein the ncAA has a reactive halide, aryl ketone, Michael acceptor, aryl isothiocyanate, or aryl carbamate side chain.

68. The composition of claim 50, wherein the ncAA has a carbamate side chain.

69. The composition of claim 68, wherein the ncAA is 4-fluorophenyl carbamate lysine (FPheK), phenyl carbamate lysine (PheK), N-acryloyl-lysine (AcrK), or 2-amino-6-(6-bromohexanamido)hexanoic acid (BrC6K).

70. The composition of claim 50, wherein the affinity compound exhibits binding affinity for the Fc region, the Fab region, or the hinge region of said antibody.

71. The composition of claim 50, wherein the affinity compound exhibits binding affinity for to the CH2 or CH3 region of said antibody.

72. The composition of claim 50, wherein the affinity compound exhibits binding affinity for to the CH2-CH3 junction of said antibody.

73. The composition of claim 56, wherein the affinity peptide has a length of 10-60 amino acids.

74. The composition of claim 56, wherein the affinity peptide has a length of 30-60 amino acids.

75. The composition of claim 74, wherein the affinity peptide has a length of 33 amino acids.

76. The composition of claim 75, wherein the affinity peptide is SEQ ID NO: 2.

77. The composition of claim 56, wherein the affinity peptide is produced by solid-phase synthesis or recombinant expression.

78. A pharmaceutical composition comprising the composition of any of claims 50-77 and a pharmaceutically acceptable buffer, diluent or excipient.

79. A method of imaging and/or treating a disease in a subject comprising administering an effective amount of a conjugated antibody of any of claims 52-77, a pharmaceutical composition of claim 78, or a conjugated antibody produced according to any of claims 1-49, to the subject.

80. A method of performing an in vitro assay comprising using a conjugated antibody of any of claims 52-77, or a conjugated antibody produced according to any of claims 1-49, to detect and/or isolate a protein.

81. The method of claim 80, wherein antibody conjugate is an antibody-HRP conjugate or antibody fluorophore conjugate.

82. The method of claim 81, wherein the assay is a western blot, flow cytometry, immunofluorescence, immunoprecipitation, or ELISA.

83. A method for producing a FPheK-labeled FB affinity peptide comprising:
(a) synthesizing a truncated FB peptide with a monomethoxytrityl (MMT) protection group using Fmoc-based solid-phase peptide synthesis;
(b) selectively removing the MMT protection group using acetic acid; and
(c) reacting the truncated FB peptide with 4-fluorophenyl chloroformate, thereby producing the FPheK-labeled FB affinity peptide.

84. The method of claim 83, wherein the MMT protection is at residue 25 of the truncated FB peptide.

85. The method of claim 83, wherein solid-phase synthesis comprises stepwise synthesis starting from rink amide resin.

86. The method of claim 83, wherein the truncated FB peptide is N-terminal acetylated.

87. The method of claim 83, wherein the acetic acid is 10% acetic acid.

88. The method of claim 83, wherein the truncated FB peptide comprises SEQ ID NO:2.

89. The method of claim 83, further comprising lyophilizing the FPheK-labeled FB affinity peptide.

90. The method of claim 83, further comprising denaturing the FPheK-labeled FB affinity peptide.

91. The method of claim 83, wherein denaturing comprises using urea.
